(19) United States
(12) Patent Application Publication
(10) Pub. No.: US 2009/0233954 A1
(43) Pub. Date: Sep. 17, 2009

(54) METHODS AND SYSTEMS FOR PROGNOSIS OF A PATIENT'S RESPONSE TO TREATMENT OF ANDROGENETIC SKIN DISORDERS

(76) Inventors: Andy Ofer GOREN, Newport Beach, CA (US); Sharon Keene, Tucson, AZ (US)

Correspondence Address: Nixon Peabody LLP, 200 Page Mill Road, Palo Alto, CA, 94306, US

(21) Appl. No.: 12/202,080

(22) Filed: Aug. 29, 2008

Related U.S. Application Data
(60) Provisional application No. 61/069,215, filed on Mar. 12, 2008.
(60) Provisional application No. 61/126,325, filed on May 1, 2008.
(60) Provisional application No. 61/060,313, filed on Jun. 10, 2008.
(60) Provisional application No. 61/078,323, filed on Jul. 3, 2008.

(51) Int. Cl. A61K 31/435; C12Q 1/68

(52) U.S. Cl. 514/284; 435/6

(57) ABSTRACT

Methods and systems are described for prediction of a patient's response to a drug for treating androgenetic skin disorders such as alopecia, acne, and hirsutism. For a given drug such as anti-androgens and 5-alpha-reductase inhibitors, and a given dosage, the likely responsiveness of that drug and dosage may be determined, for example, by generating a set of patient scores based on a patient profile, including genetic sample information, and comparing that scores to a set of reference information.

### CROSS-REFERENCE TO RELATED APPLICATIONS

This application claims the benefit of priority from U.S. Provisional Application No. 61/069,215 filed Mar. 12, 2008, entitled “System and Method for Diagnosing Female Alopecia”; U.S. Provisional Application No. 61/126,325 filed May 1, 2008, entitled “System and Method for Treatment of Female Androgenetic Alopecia Based on Genetic Screening”; U.S. Provisional Application No. 61/060,313 filed Jun. 10, 2008, entitled “System and Method for Treatment of Female Androgenetic Alopecia Based on Genetic Screening”; and U.S. Provisional Application No. 61/078,323 filed Jul. 3, 2008, entitled “System and Method for Treatment of Acne and Related Skin Disorders Based on Genetic Screening.”

### CROSS-REFERENCE TO SEQUENCE LISTING

The sequence listing included in the electronic file submitted herewith via EFS-Web as one of the parts of this application, entitled “Sequence_Listing_{—}60374_{—}14.txt”, is incorporated by reference into this application in its entirety.

### TECHNICAL FIELD

This subject matter relates generally to methods, information systems, and diagnostic kits for the treatment of androgenetic skin disorders on the basis of a patient's genetic information or other patient criteria.

### BACKGROUND

Androgenetic skin disorders are associated with a variety of medical, psychological, and social implications, and there have been many efforts to find effective ways to treat some of these disorders, with mixed success. Perhaps the most well-known such disorder, androgenetic alopecia (hair loss), has a number of effective treatments, but the results have been mixed. Moreover, the hair loss industry is littered with dozens of products that claim to grow, improve, and replace hair. Unfortunately, few treatments have been scientifically demonstrated to work, and the few treatments that have undergone clinical trials often do not work equally for all patients.

Androgenetic alopecia has been successfully treated in men by the U.S. Food & Drug Administration (“FDA”) approved medications minoxidil (marketed as Rogaine™ or Regaine™) and finasteride (marketed as Propecia™, among other brand names). Minoxidil has also been approved by the FDA for treatment of female hair loss; however, for most women minoxidil is only marginally successful at retaining existing hair. Some men for whom minoxidil is less effective have been successfully treated with finasteride; however, the same cannot be said of females for whom minoxidil is ineffective. Studies have thus far failed to show the effectiveness of finasteride in the treatment of female androgenetic alopecia. Studies have also thus far failed to show the effectiveness of finasteride in low dosage forms less than about 1 mg.

There are significant differences between male androgenetic alopecia and female androgenetic alopecia. Apart from the different baldness patterns, male and female alopecia follow a different mechanism. In men, alopecia is related to the normal high androgen levels in males, combined with an underlying sensitivity of the hair roots to androgens. Women, however, have roughly 10 times lower androgen levels than men, and the absolute amount of androgen is a less significant factor than the increased sensitivity of the hair roots to androgens.

Because of the inherent differences between men and women, presumed to be genetic, researchers have not thus far been successful in treating females using methods previously found to be effective in males. It has been generally thought, for example, that finasteride is ineffective as a general-purpose treatment of female androgenetic alopecia. It would therefore be advantageous to be able to have an effective diagnostic and treatment method where female patients could be selected and treated on the basis of criteria such as genetics, which would allow at least some women to he identified and effectively treated with known alopecia treatments, or any treatments that may be discovered in the future. Similarly, the same selection and treatment methods could be used to treat male androgenetic alopecia using lower dosages than are presently thought to be effective. Treatment with lower dosages may be advantageous in reducing undesired side effects.

Like alopecia, adult onset acne is associated with a variety of psychological and social implications. Topical agents are first used to treat adult onset acne; however, patients that suffer from moderate to severe acne, acne that scars, or acne that is not responsive to topical treatments require systemic therapy. Systemic treatments are divided into two categories: antibiotics and hormonal therapies. The most common systemic antibiotics used for the treatment of acne are tetracyclines. Tetracyclines have several side effects such as staining teeth, increased risk of sun burns, and increased risk for vulvovaginitis in women. Other antibiotic treatments have various side effects as well as different response profiles. An alternative, or some times adjuvant, systemic therapy to antibiotics are hormonal therapies. Hormonal therapies are used on women that are treatment-resistant to antibiotics, or when an abnormal androgenetic endocrine disorder is suspected such as: ovarian cysts, infertility, hyperandrogenism, irregular menstrual cycles, or acne associated with hirsutism. In addition, long term use of antibiotics in women is not recommended by physicians and thus hormonal therapies are often used for the treatment of adult onset acne in women. Among the hormonal treatments, anti-androgens are often prescribed for adult onset acne in females; however, some patients fail to respond to the anti-androgen therapy often presenting severe side effects such as dry skin, hot flashes, increased appetite, and decreased libido.

Currently physicians treating women with adult onset acne do not have a method to predict the efficacy or the side effects of different systemic therapies; hence, physicians often prescribe patients treatments that result in little or no improvement in the patient's acne condition and may cause severe side effects. To complicate matters, the treatments are typically prescribed for a period of 3 to 6 months before the physician may try a different treatment on the patient.

It would therefore be advantageous if a patient's response to different acne treatments could be predicted and thus reduce side effects, improve patient outcome, and reduce treatment cost. In accordance with the disclosure herein, use of a patient's androgen receptor genetic variations is made to identify the anti-androgen treatments that are most likely to improve a patent's acne condition.

### BRIEF SUMMARY

The present disclosure relates to methods and systems for prognosis of a patient's response to a drug for treating an androgenetic skin disorder. Various embodiments are possible, a number of which are exemplified here.

In one embodiment of the present disclosure, there is provided a method comprising obtaining a sample, extracting genetic material, generating a genetic profile from the genetic material comprising a set of patient score values, each of which may be a function of one or more measurable characteristics of the genetic material, and generating a prognosis as to whether or not the patient is likely to be responsive to the drug at a selected dosage, based on a comparison between patient score values and reference information.

In another embodiment, there is provided a system comprising means for storing a genetic profile from a patient, means for storing reference information comprising aggregate data from a plurality of reference profiles from patients with positive and negative responses to the drug, means for comparing patient profiles with the reference information, and means for generating a prognosis as to whether or not the patient is likely to be responsive to the drug at a selected dosage.

In both of the above embodiments, the measurable characteristics and reference characteristics may include the genotype of an AR gene polymorphism within the genetic material, the genotype of a polymorphism in linkage disequilibrium with an allele of an AR gene polymorphism within the genetic material, the genotype of a 5-alpha-reductase gene polymorphism within the genetic material, the genotype of a polymorphism in linkage disequilibrium with an allele of a 5-alpha-reductase gene polymorphism within the genetic material, a measured concentration of 5-alpha-reductase within the sample, and a measurement of transcriptional activity of the 5-alpha-reductase gene within the sample.

### BRIEF DESCRIPTION OF THE DRAWINGS

The accompanying drawings, which are incorporated into and constitute a part of this specification, illustrate one or more exemplary embodiments of the inventions disclosed herein and, together with the detailed description, serve to explain the principles and exemplary implementations of these inventions. One of skill in the art will understand that the drawings are illustrative only, and that what is depicted therein may be adapted based on the text of the specification or the common knowledge within this field.

In the drawings:

FIG. 1 shows a method for generating a prognosis as to whether a drug will likely be effective at a given dosage to treat a given androgenetic skin disorder.

FIG. 2 shows a system for generating a prognosis as to whether a drug will likely be effective at a given dosage to treat a given androgenetic skin disorder.

### DETAILED DESCRIPTION

Various example embodiments of the present inventions are described herein in the context of the treatment of male and female androgenetic skin disorders on the basis of a patient's genetic information or other patient criteria. Example embodiments are disclosed, including methods and systems for prognosis of a patient's response to a drug for treating an androgenetic skin disorder. Such embodiments have the advantage that they allow the effective treatment of androgenetic skin disorders by identifying and screening patients for which particular treatments are likely to be effective.

The inventions disclosed herein exploit a number of inventive insights, including without limitation the insight that treatments for androgenetic skin disorders which have not heretofore been considered treatable with particular drugs may be treated effectively through application of the present disclosure and associated claims.

Those of ordinary skill in the art will understand that the following detailed description is illustrative only and is not intended to be in any way limiting. Other embodiments of the present inventions will readily suggest themselves to such skilled persons having the benefit of this disclosure, in light of what is known in the relevant arts, such as the arts of medical treatment, the provision and operation of information systems for such treatment, and other related areas. Reference will now be made in detail to exemplary implementations of the present inventions as illustrated in the accompanying drawings.

In the interest of clarity, not all of the routine features of the exemplary implementations described herein are shown and described. It will of course, be appreciated that in the development of any such actual implementation numerous implementation-specific decisions must be made in order to achieve the specific goals of the developer, such as compliance with regulatory, safety, social, and business-related constraints, and that these specific goals will vary from one implementation to another and from one developer to another. Moreover, it will be appreciated that such a developmental effort might be complex and time-consuming, but would nevertheless be a routine undertaking of engineering for those of ordinary skill in the art having the benefit of this disclosure.

Throughout the present disclosure, relevant terms are to be understood consistently with their typical meanings established in the relevant art. However, without limiting the scope of the present disclosure, further clarifications and descriptions are provided for relevant terms and concepts as set forth below:

The term DHT refers to dihydrotestosterone.

The term androgen receptor (AR) has its ordinary meaning within the field, and refers to the class of nuclear receptors within living cells characterized by binding to several chemical or biological compounds including without limitation testosterone, DHT, and/or progestins.

The term androgenic hormone refers to a hormone that binds to an androgen receptor, including without limitation testosterone and/or DHT.

The term androgenetic alopecia refers to forms of hair loss that occur under the influence of androgenic hormones in connection with a genetic disposition. These forms of hair loss are generally associated with an acceleration of the hair cycle, which leads to the proliferation of hair in the rest phase of the cycle and increasing occurrence of thin miniature hairs.

The term androgenetic skin disorder refers to any disorder of the skin related to a disturbance in the normal functioning of androgenic hormones. It includes, without limitation, androgenetic alopecia, adult onset acne, and hirsutism.

The term polymorphism has its ordinary meaning in the field, and broadly includes any difference in the genome sequence among individuals of the same species. A “single nucleotide polymorphism” (SNP) refers to a polymorphism at a single position.

The term gene has its ordinary meaning in the field, and broadly includes any segment of DNA associated with a biological function, or any other genetic material, such as RNA, that corresponds to or copies the information found in such DNA. It includes coding sequences, regulatory sequences, and non-expressed sequences that may, for example form recognition sequences for proteins. A gene may be obtained from a variety of sources, including extraction from a biological source, cloning, or synthesis.

The term genetic material has its ordinary meaning in the field, and may refer, without limitation, to any gene or other sequence of nucleic acids, including DNA or RNA in any form, whether originating in a cell nucleus, the cytoplasm, mitochondria, or otherwise, or which are synthesized or produced by prokaryotes, and may include nucleic acids that are copies of other genetic material.

The term 5-alpha-reductase refers to a class of enzymes in the body that convert testosterone to DHT. There is more than one 5-alpha-reductase enzyme, including without limitation SRD5A1 and SRD5A2, and reference to this term refers to each of the 5-alpha-reductase enzymes, and all of them.

The term 5-alpha-reductase gene refers to any gene or other genetic material for 5-alpha-reductase, including without limitation genes for SRD5A1 and SRD5A2.

The term 5-alpha-reductase inhibitor refers to any substance that inhibits the action or availability of 5-alpha-reductase, including without limitation finasteride (Propecia™), duasteride, FCE 28260, and saw palmetto. The term may also include further classes of compounds that may be found through further research to be effective in inhibiting or suppressing 5-alpha-reductase by any mechanism, whether presently known or unknown.

The term 5-alpha-reductase polymorphism refers to any polymorphism on any 5-alpha-reductase gene, or in linkage disequilibrium with another 5-alpha-reductase polymorphism. This may include, without limitation, the repeated TA microsatellite, found in the 3′ untranslated region of the SRD5A2 gene, and any genetic sequence that is in linkage disequilibrium with an allele of this microsatellite. It may also include any SNP in either SRD5A1 or SRD5A2, or any genetic sequence in linkage disequilibrium with an allele of such SNP.

The term anti-androgen refers to any substance that directly or indirectly inhibits the production or effect of an androgenic hormone, including without limitation 5-alpha-reductase inhibitors, spironolactone, flutamide, cyproterone acetate, nilutamide, and bicalutamide. This may also include, without limitation, any agent that interferes with the DHT or testosterone androgen pathway.

The term androgen-related gene refers to any gene that is directly or indirectly regulated by an androgen, that directly or indirectly regulates an androgen, that is expressed in direct or inverse correlation with another androgen-related gene. This includes, without limitation, an AR gene, and an 5-alpha-reductase gene.

The term androgen-related polymorphism refers to any polymorphism in an androgen-related gene. This includes, without limitation, an AR gene polymorphism and an 5-alpha-reductase polymorphism.

The term AR gene refers to any gene or other genetic material for an androgen receptor.

The term AR CAG microsatellite refers to the CAG microsatellite in the first exon of an AR gene, located at around codon 58.

The term AR GGN microsatellite refers to the GGN microsatellite at around codon 442 of an AR gene, where N is A, C, G, or T, and most preferably, N is C, G, or T.

The term AR gene polymorphism refers to any polymorphism on an AR gene, or in linkage disequilibrium with another AR gene polymorphism. This may include, without limitation, the AR CAG microsatellite, the GGN microsatellite, the SNP rs6152, and any genetic sequence that is in linkage disequilibrium with an allele of the AR CAG microsatellite, the GGN microsatellite, the SNP rs6152, or another AR gene polymorphism.

The term EDA2R gene refers to any gene or other genetic material for an ectodysplasin A2 receptor.

The term EDA2R gene polymorphism refers to any polymorphism on an EDA2R gene, or in linkage disequilibrium with another EDA2R polymorphism. This may include, without limitation, SNP rs1385699, and any genetic sequence that is in linkage disequilibrium with an allele of SNP rs1385699 or another EDA2R polymorphism.

The terms medium and media refer to any tangible means of storing information, including any apparatus or computer in which the information can be stored. This may include, without limitation, papers, graphs, permanent or temporary electronic storage media of any kind, memories, integrated circuits capable of storage, optical disks, hard disks, or neural networks. The term medium may refer to one or more media used in conjunction with each other, or media where information can be transmitted between different intermediate storage locations or states. A neural network can be a medium for storage, among other things, of its training.

The term aggregate data means data that has been compiled, collected, or synthesized from a plurality of sources. Aggregate data may include, without limitation, data that has been averaged, correlated, or placed unchanged within a database.

The term number, in relation to reference information and patient scores according to this disclosure, means a data value that can be compared to a second data value within the same relevant set of potential data values, and for which the data value can be said to be less than, greater than, or equal to the second data value. A number in this context may be, without limitation, an integer, complex, or a real number.

The term drug is used herein according to its normal and ordinary meaning, and additionally includes any chemical or biological substance, or combination of substances, that may be administered at a specific dosage at a specified frequency as a form of treatment or therapy, or as part of an investigation for possible use in treatment or therapy, or to purposely provoke a desired physiological effect. A drug may be a solid, liquid, or a gas, and may be administered orally, intravenously, topically, by inhalation, or otherwise. A drug may comprise either a single active ingredient or a plurality of active ingredients, and may additionally contain inert or non-therapeutic ingredients, or ingredients that have a separate therapeutic value. Anti-androgens and 5-alpha-reductase inhibitors are two of many classes of drugs.

The term patient is used herein according to its normal and ordinary meaning, and additionally refers to the subject of any treatment or therapy, or any experimental or investigational study as to the viability of any treatment or therapy. A patient may include a human or a non-human animal. It may also include reference patients from whom data has been collected. Status as a patient does not necessarily require the existence of a physician or health care provider; for example, a patient may self-administer a treatment or therapy.

The terms responsive or responsiveness, when used in reference to a drug and a patient, describes the situation where a drug has succeeded in inducing an intended therapeutic or physiological effect. For example, a drug treatment for an androgenetic skin disorder is responsive in a patient if the patient's disorder has been measurably or subjectively slowed, halted, or reversed, using criteria known in the art. For example, responsiveness with respect to androgenetic alopecia may be determined by, without limitation: a subject's self-assessment of his or her hair, an investigator's assessment using a standardized scale, a global photography assessment of a patient's skin or hair, or criteria such as whether the patient's hair has changed with respect to hair growth, hair thickness, selected hair diameter, average hair diameter, or average hair length.

In certain cells in various organs of the body, particularly in hair follicles and sebaceous glands within the skin, testosterone can be converted to DHT through the action of 5-alpha-reductase. DHT then binds with the androgen receptor (AR) in the cell nucleus, and the DHT-AR complex dimerizes and then interacts with particular nuclear DNA sequences to either up- or down-regulate a specific gene transcription. Such AR gene expression plays a role in androgenetic skin disorders such as alopecia, acne, and hirsutism.

The gene encoding AR is found on the X-chromosome. Encoded in exon 1 of the AR gene is a polymorphic region containing a CAG microsatellite. The number of CAG repeat units within this microsatellite varies within a wide range, and in normal individuals might range from roughly 10 to 30. This length may also vary based on ethnicity. Higher numbers of repeats, greater than about 40, are known to be linked to Kennedy's disease, also known as X-linked spinal and bulbar muscular atrophy (SBMA). Other diseases, including various cancers and male infertility, are known to be associated with the length of the CAG microsatellite.

The CAG microsatellite encodes for a polyglutamate chain in the transactivation region of the AR, at around codon 58. The length of this polyglutamine chain has been shown to correlate inversely with transactivation of the AR. See Chamberlain, N. L., et al., Nucleic Acids Res., 22(15): 3181-3186 (1994).

In addition to the CAG microsatellite, exon 1 of the AR gene contains another microsatellite at around codon 442, containing repeated GGN units, where N may be A, C, G, or T, which codes for polyglycine. The majority of these codons are known to be GGC. As with the CAG microsatellite, there is thought to be an inverse correlation between the length of the GGN microsatellite and the transactivation of the AR. See Ding D. et al., Prostate, 62: 133-139 (2005).

The first exon of the AR gene also contains SNP rs6152, which is known to be strongly correlated with male pattern baldness. See Ellis et al., Hum. Genet., 121(3-4): 451-457 (2007). Another SNP that may be related to baldness is SNP rs1385699, which resides on the EDA2R gene. There may also be polymorphic regions of the AR, EDA2R, and other genes that affect androgenetic skin disorders. Moreover, polymorphic regions affecting one androgenetic skin disorder, such as male androgenetic alopecia, may affect other androgenetic skin disorders.

Further, there are a number of polymorphisms on the 5-alpha-reductase genes, such as a repeated TA microsatellite found in the 3′ untranslated region of the SRD5A2 gene, and there are various other polymorphisms and SNPs on the SRD5A1 and SRD5A2 genes. These polymorphisms, and most preferably those within the SRD5A1 gene, may correlate with 5-alpha-reductase activity, and thus affect androgenetic skin disorders.

Many other genetic polymorphisms may affect a patient's ability to respond to a given drug for treatment of androgenetic skin disorders. The likely response may also be affected by other factors, including the measured concentration of 5-alpha-reductase within the sample, or the measurement of transcriptional activity of a 5-alpha-reductase gene with the sample. A particular allele of a 5-alpha-reductase gene polymorphism may correspond to an increase or decrease in such measured concentrations and/or activity.

Preferably, the determination of whether a patient is likely to respond to a given drug for treatment of androgenetic skin disorders may be based on a number of genetic factors, and other factors such as, without limitation, the patient's racial profile, the patient's medical history, or the medical history of the patient's ancestors or relatives. Each of these factors may be combined, as part of a mathematical formula, an algorithm, or a neural network, to obtain a prognosis of a patient's likely response to a given drug.

The present disclosure provides, among other embodiments, and teachings, a method for prognosis of a patient's response to a drug for treating an androgenetic skin disorder, comprising: obtaining a sample from the patient comprising one or more cells; extracting genetic material from the sample; generating a patient profile; and generating a prognosis as to whether or not the patient is likely to be responsive to the drug at a selected dosage, wherein the step comprises comparing one or more of the patient score values with reference information stored in one or more media. The patient profile may comprise a set of one or more patient score values, each patient score value being a function of one or more measurable characteristics. These measurable characteristics can include one or more genotypes within the genetic material, where each genotype is an allele of a genetic polymorphism such as an androgen-related polymorphism, an EDA2R gene polymorphism, or a polymorphism having an allele in linkage disequilibrium with an allele of a genetic polymorphism such as an androgen-related polymorphism or an EDA2R gene polymorphism. The measurable characteristics may also include a measured concentration of 5-alpha-reductase within the sample, or a measurement of transcriptional activity of an 5-alpha-reductase gene within the sample.

Preferably, each of the one or more genotypes may be an allele of a genetic polymorphism such as an AR gene polymorphism, a 5-alpha-reductase gene polymorphism within the genetic material, or an EDA2R gene polymorphism. Most preferably, the genotypes may be an AR CAG microsatellite, an AR GGN microsatellite, SNP rs6152, or SNP rs1385699. In addition, any polymorphism may be used according to the present disclosure where there is a direct or indirect correlation between the androgenetic skin disorder and the gene, or a polymorphism on the gene, or a polymorphism in linkage disequilibrium with a polymorphism on the gene. Particular polymorphism targets may include genes that are particularly expressed in hair follicles and sebaceous glands, and preferably those genes that relate to the regulation or reception of androgens.

A patient score value may be a function of one or more measurable characteristics in a number of ways which will be apparent to one of skill in the art in view of this disclosure. For example, in one embodiment, a patient score might the length of the CAG microsatellite, and thus be a function of an allele of the CAG microsatellite polymorphism. In another embodiment, a patient score value may be 1 or some other data value if one particular allele is present, and 0 or some other data value if the allele is absent. Data values or numbers other than 1 or 0 might be used, including mathematical constructs that are not numbers. The data values may, optionally, be stored or converted into binary form in a computer medium. In yet another embodiment, a patient score that is a function of the allele of an SNP may be 0, 1, 2, or some other number or data value, each data value corresponding to a particular allele of the SNP. A patient score value may also, for example, be a mathematical sum, product, average, ratio, comparison, or other combined function of one or more numbers or data values such as those described above.

One embodiment of the present disclosure relates to the discovery that, in females, a common Androgen Receptor (AR) variant, a variable CAG repeat length in the first exon, causes changes in the hair follicle's response to DHT. The hormone DHT can be formed by the interaction of 5-alpha-reductase with testosterone, the main androgen or “male hormone.” In predisposed follicles, exposure to DHT causes shortening of the anagen (growth) phase of the hair cycle. As hair growth time becomes shorter, hairs gradually become smaller and finer. This leads to the emergence of vellus hairs, or “peach fuzz.” Eventually, hairs stop growing altogether. Unlike in men, in women, androgenetic alopecia does not follow a distinct pattern, but appears as a diffused hair thinning.

In females, androgenetic alopecia is extremely common, affecting over 50% of females by the age of 60. The risk for female androgenetic alopecia can be predicted by examining variations in the Androgen Receptor gene. Unfortunately, studies have shown that therapies that are successful at hair re-growth and maintenance in males failed to show significant improvement in females. One aim of this invention is to screen women that will respond to certain therapy and thereby provide personalized effective therapy for hair loss.

Clinical trials with finasteride for the treatment of female androgenetic alopecia have shown that finasteride's affect on hair re-growth in women was statistically not significant; however, physicians prescribing finasteride to women often observe significant hair re-growth in some women. The current disclosure provides, among other things, methods and systems of using genetic variations in the AR gene as drug response markers for female androgenetic alopecia. Based on genotyping of a female patient's CAG and/or GGN repeat lengths in the first exon of the Androgen Receptor, or other AR gene polymorphisms, or polymorphisms of the 5-alpha-reductase gene, or measuring 5-alpha-reductase concentration or transcription activity in the 5-alpha-reductase gene, the methods and systems disclosed herein allows a physician to select the appropriate medication and dosage for treatment of female androgenetic alopecia.

In addition to treating women with finasteride, it may also be possible to treat androgenetic alopecia in males according the present disclosure using dosages of finasteride which have not heretofore been found effective in males, such as dosages less than 1.0 mg per day, and preferably 0.5 mg per day. The present invention can be used to generate a prognosis regarding various dosages of a given drug, based on the patient's genetic profile. Without limitation, a prognosis may be generated as to whether a treatment of a particular male with a particular dosage such as 0.5 mg per day would likely be effective. Similarly, without limitation, a prognosis may be generated as to whether a treatment of a particular female with a particular dosage such as preferably 2.5 mg per day or less or, for example, 1.0 mg per day, would likely be effective.

The inventions disclosed herein can be used for treatment of androgenetic skin disorders using drugs other than finasteride, such as without limitation anti-androgens, and preferably 5-alpha-reductase inhibitors, including any drug that is a composition that inhibits the activity of the DHT or testosterone androgen pathway.

The inventions may apply to other diseases than androgenetic alopecia, including adult onset acne and hirsutism. In particular, adult onset acne (sometimes referred to as hormonal acne) is common, especially in women, affecting over 50% of women between the age of 20 and 30 and over 25% of women between the age of 40 and 50. Scientific studies have revealed that variations in the androgen receptor gene are associated with acne, hirsutism, and other skin conditions.

One embodiment of the present disclosure relies on the discovery that in females, different AR gene polymorphisms are associated with sebaceous gland response to DHT. In genetically predisposed sebaceous glands, prolonged exposure to androgens and particularly DHT causes increased sebum production and ultimately the formation of severe acne. Due to the predisposing genetic variations, often, patients with normal androgen levels experience adult onset acne.

Clinical studies and observations using anti-androgens such as but not limited to spironolactone, flutamide, finasteride, cyproterone acetate, and dutasteride are often effective at the treatment of adult onset acne in women; however, response to anti-androgens can take up to several months (typically 3 or 6 months) during which time patients may suffer side effects, possibly with no ultimate clinical benefit. The current invention provides a method of using genetic variations in the first exon of the androgen receptor gene as drug response markers for the treatment of adult onset female acne. Based on genotyping of a female patient's CAG and/or GGN repeat lengths in the first exon of the androgen receptor gene, the system disclosed herein allows a physician to select the appropriate medication and dosage for the treatment of adult onset female acne, and/or to predict whether a particular treatment should be undertaken at all.

In accordance with one approach described herein, which is not intended to be limiting, a patient's genetic sample may be obtained. The sample may be genotyped for the CAG repeat length in the first exon of the Androgen Receptor gene (AR) using various methods known in the art, such as PCR or nucleotide sequencing. For a female patient who has two copies of the AR gene, one on each X chrosomome, CAG repeat length for each copy of the AR gene may be obtained. The average CAG repeat length can be calculated as the patient's CAG score. If the CAG score is below a certain threshold such as, preferably, 20, there may be an increased likelihood that the patient will be responsive to finasteride for treatment of androgenetic skin disorders. In an alternative embodiment, the patient's CAG score may be obtained by selecting the longer of the CAG repeat lengths. In yet another alternative embodiment, the patient's CAG score may be obtained by selecting the repeat length from the AR gene located on the activated X chromosome of the female patient.

In yet another embodiment, the patient's GGN repeat length may be obtained by genotyping one or more homologous copies of the AR gene in a female patient, using similar means for the determination of the CAG repeat length, as discussed above and as known in the art. Thus, the CAG and GGN repeat length for each copy of the AR gene may be obtained. The combined average repeat length can be calculated as the patient's score. If the score is below some threshold, preferably 40, there may be an increased likelihood that the patient will be responsive to finasteride for hair re-growth or retention. In an alternative embodiment, the patient's score may be obtained by selecting the longer of the combined repeat lengths. In yet another alternative embodiment, the patient's score may be obtained by selecting the combined repeat lengths from the AR gene located on the activated X chromosome of the female patient. In yet an alternative embodiment, the GGN repeat length may be used to determine finasteride responsiveness on female patients without use of the CAG repeat length. In this embodiment, the system disclosed herein may provide a physician with information indicative of a better response to finasteride for treatment of androgenetic skin disorders in patients with shorter GGN repeat length than a specified threshold, preferably 20.

The sample from the patient can be obtained in a number of ways. The patient can provide, as non-limiting examples, buccal cells collected by a cheek swab, blood samples, or samples from the patient's integumentary system, including hair and/or follicle samples or biopsies of affected skin, such as scalp skin. These samples could be obtained by a physician or by the patient using a sample collection kit. For example, a kit might be sent to the patient through the mail containing instructions for collecting a cheek swab, and the swab, with the patient's genetic material might be mailed to a central location where the patient's genetic profile might be obtained. Similar kits might also be provided for hair samples or other genetic samples.

As another example, a patient's genetic profile might comprise a value associated with the amount or concentration of active 5-alpha-reductase in the target tissue, patient's scalp or hair stem cells, or the transcription activity of the gene for 5-alpha-reductase in such cells, and such cells might be obtained through a biopsy performed by a physician.

While genetic analysis of a patient's genomic DNA is preferred, the present disclosure could also be practiced using other genetic material such as RNA or cDNA, or cultures of a person's DNA. A patient's genetic material may be amplified and/or copied, as well, using various methods known in the art such as polymerase chain reaction (PCR).

To obtain a genetic profile, many methods may be used which are known in the art, including PCR, DNA sequencing, or hybridization to allele-specific oligonucleotide probes, which might use substrates such as DNA microarrays or beads. Appropriate primers for various polymorphic regions in the AR and in 5-alpha-reductase are known in the art. For example, appropriate primers for the AR CAG microsatellite might be the forward primer 5′-GTG CGC GAA GTG ATC CAG A-3′ (SEQ ID NO: 1), and the reverse primer 5′-GTT TCC TCA TCC AGG ACC AGG TA-3′ (SEQ ID NO: 2). Appropriate primers for the AR GGN microsatellite, and wherein the PCR amplification might be the forward primer 5′-CCG CTT CCT CAT CCT GGC ACA C-3′ (SEQ ID NO: 3), and the reverse primer 5′-GCC GCC AGG GTA CCA CAC ATC-3′ (SEQ ID NO: 4). In addition, proteins may be used to bind specifically to particular polymorphic regions using mechanisms known in the art. See, e.g., Yano-Yanigisawa et al, Nucleic Acids Res., 23:2654-60 (1995).

Among the polymorphic regions that may be used according the present disclosure, without limitation, one might select polymorphisms from the AR CAG microsatellite, the AR GGN microsatellite, SNP rs6152 within the AR gene, SNP rs1385699 within the EDA2R gene, or the TA dinucleotide microsatellite within the SRD5A2 5-alpha-reductase gene. There may be other such polymorphic regions on these and other genes. One might also select any other genetic sequence that is in linkage disequilibrium with an allele of one of the above polymorphisms.

In an embodiment of the inventions disclosed herein, the reference information may comprise aggregate data from a plurality of reference profiles, each reference profile corresponding to a reference patient who has been administered the drug, and from whom a reference sample has been collected. Each reference profile may include, among other things, an indication of the corresponding reference patient's responsiveness or non-responsiveness to the drug. A reference profile may also include a set of one or more reference values, each reference value being a function of one or more measurable reference characteristics. These measurable reference characteristics may include one or more reference genotypes within genetic material extracted from the reference sample, each reference genotype being an allele of a genetic polymorphism such as an androgen-related polymorphism, an EDA2R gene polymorphism, or a polymorphism having an allele in linkage disequilibrium with an allele of a genetic polymorphism such as an androgen-related polymorphism and an EDA2R gene polymorphism. The measurable characteristics may also include a measured concentration of 5-alpha-reductase within the a reference sample, or a measurement of transcriptional activity of an 5-alpha-reductase gene within the reference sample. The plurality of reference profiles may include a first reference profile corresponding to a first reference patient whose reference profile is indicative of responsiveness to the drug, and a second reference profile corresponding to a second reference patient whose reference profile is indicative of non-responsiveness to the drug.

To create a genetic profile for a patient, or for a reference patient, one might use one, two, three, or any number of the measurable characteristics such as discussed above, or all of them It is preferable to use as many measurable characteristics as possible, particularly those for which there may be a correlation between the measurable characteristic and the prognosis. Such correlation may be performed using various statistical or computational methods known in the art, such as, for example, linear regression, linear programming, optimization, or use of neural networks which have been trained using reference patient profiles.

The reference information may contain several types of information, including genetic data. However, the reference information is not limited to genetic information, and may contain indications of responsiveness or non-responsiveness, or various other information, such as a patient's self-assessment, a physician's assessments regarding the patient's responsiveness to a treatment, photographs of the patient or the patient's hair or skin, evaluations of criteria such as, without limitation, whether the hair of a patient undergoing treatment for androgenetic alopecia has changed with respect to hair growth, hair thickness, selected hair diameter, average hair diameter, or average hair length. The reference information may also include racial, ethnic, or other population-related characteristics, information about the patient's medical history, or the medical history of the patient's ancestors or relatives.

The patient's genetic profile may be compared to a reference profile or other set of reference information, which might include aggregate information from various reference patients. Such information may be aggregated in many ways, including averaging, correlation, the creation of predictive linear or non-linear mathematical formulas. The reference profile may, in a preferred embodiment, consist of a single number for comparison with a genetic trait by the patient, such as without limitation the length of the CAG, GGN, or TA microsatellite, or a number such as 0, 1, 2, or some other number corresponding to the particular allele within a SNP such as without limitation SNP rs6152 or SNP rs1385699. Alternatively, a number or data value such as 1 may indicate the presence of a particular allele, and 0 or some other number or value may indicate its absence. Aggregation may also include simply storing the raw data in a database for direct comparison with the patient's genetic profile. Aggregate data may comprise a statistical mean, media, or mode, or be based on a statistical distribution of patient reference values, and may preferably include a set of values, such as without limitation mean and standard deviation, sufficient to describe a statistical distribution, such as a normal distribution. One may determine a patient score using the methods disclosed herein, and compare that with a statistical distribution of reference scores. One may, for example, make a determination as to whether the patient will be responsive to the drug by comparing a patient score with the median, mode, or average of patient scores, or by determining the distance from the mean, which may preferably be measured in the number of standard deviations in a normal distribution. The comparison may also be based on other statistical criteria known in the art. Comparison between the patient's profile and the reference information can be performed directly by a researcher, physician, medical professional, or may be automated by a computer.

In a preferred embodiment, generating a prognosis may include generating either of two or more alternate comparison results, the first result indicating that the patient is likely to be responsive to the drug, and the second result indicating that the patient is not likely to be responsive to the drug. A third result might indicate inconclusiveness, or preferably, any indication of inconclusiveness might be included as part of the second result. Each of the comparison results might take many forms, including without limitation a signal, a transmission, a value stored on a medium or computer memory, or a written indication of likely responsiveness. Additionally, one of the comparison results might also consist merely of the absence of one or more of the other the comparison results.

In a further embodiment, the methods disclosed herein might include administration of the drug to the patient, providing a prescription for the drug to the patient so that the patient may self-administer the drug, or it might involve recording the prognosis on a medium and/or transmission or delivery to the patient, which might occur in many ways, such as for example by mail, electronically, in a face-to-face meeting between the patient and the doctor, or by interactive means such as through a website.

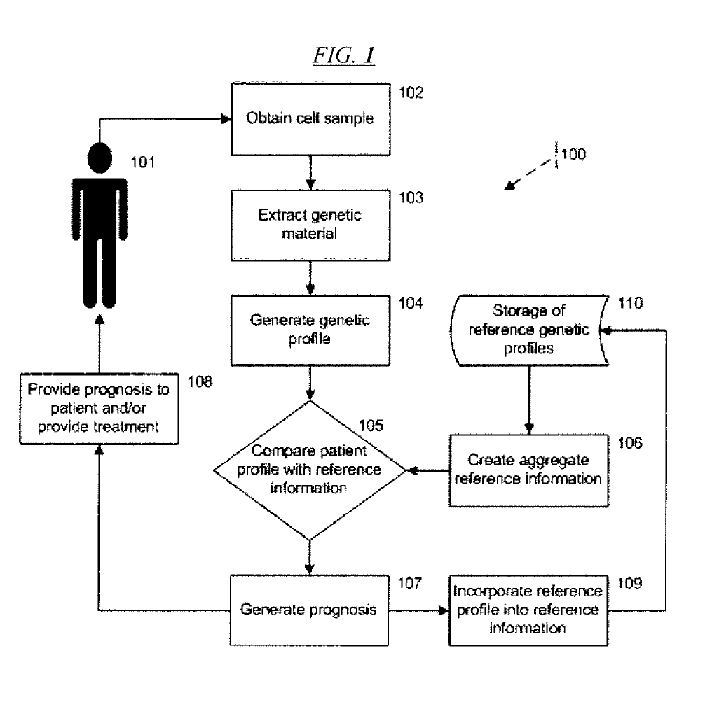

As an example, with reference to FIG. 1, a method and system 100 is described showing a patient 101. A cell sample may be obtained 102 from the patient, genetic material may be extracted 103, a genetic profile may be obtained 104, and compared with aggregate reference information which has been aggregated 106 from stored reference profiles 110. The patient's genetic profile may be compared 105 with the reference information to generate a prognosis 107. That prognosis may be incorporated 109 into the stored reference profiles 110. The prognosis may also be provided to the patient, and the patient might optionally be treated or provided with a prescription for the drug.

Optionally, a patient's genetic profile may be associated with a unique identifier. A patient might access a prognosis by using that unique identifier or other security information on a website. Alternatively, the prognosis might be given through a caregiver. Various information might be included as part of the prognosis, including without limitation an indication of the likelihood that the patient will be responsive to finasteride or another drug, a prediction of the dosage required, an indication of the likelihood that the patient will experience side effects to the drug, the severity, pattern, and time of onset of an androgenetic skin disorder.

As a further embodiment of the inventions disclosed herein, there is provided a system for generating a prognosis of a patient's response to a drug for treating an androgenetic skin disorder. This system may include means for storing a patient profile comprising a set of one or more patient score values, each patient score value being a function of one or more measurable characteristics. These measurable characteristics may include, among other things, one or more genotypes within the genetic material, each genotype being an allele of a genetic polymorphism such as an androgen-related polymorphism, an EDA2R gene polymorphism, or a polymorphism having an allele in linkage disequilibrium with an allele of a genetic polymorphism such as an androgen-related polymorphism or an EDA2R gene polymorphism. The measurable characteristics may also include a measured concentration of 5-alpha-reductase within the sample, or a measurement of transcriptional activity of an 5-alpha-reductase gene within the sample.

The system may also include means for storing reference information comprising aggregate data from a plurality of reference profiles, each reference profile corresponding to a reference patient who has been administered the drug, and from whom a reference sample has been collected. Each reference profile may include, among other things, an indication of the corresponding reference patient's responsiveness or non-responsiveness to the drug, and a set of one or more reference values, each reference value being a function of one or more measurable reference characteristics. These measurable reference characteristics may include one or more reference genotypes within genetic material obtained extracted from the reference sample, each reference genotype being an allele of a genetic polymorphism such as an androgen-related polymorphism, an EDA2R gene polymorphism, and a polymorphism having an allele in linkage disequilibrium with an allele of a genetic polymorphism such as an androgen-related polymorphism or an EDA2R gene polymorphism. Other measurable reference characteristics might include a measured concentration of 5-alpha-reductase within the reference sample, or a measurement of transcriptional activity of an 5-alpha-reductase gene within the reference sample. The reference profiles may include a first reference profile corresponding to a first reference patient whose reference profile is indicative of responsiveness to the drug, and a second reference profile corresponding to a second reference patient whose reference profile is indicative of non-responsiveness to the drug.

The system described above may also include means for comparing one or more of the patient score values with the reference information, and means for generating a prognosis as to whether or not the patient is likely to be responsive to the drug at a selected dosage.

All the considerations for the methods described in this disclosure are also applicable to the disclosed system. Preferably, the genotypes may be an allele of a genetic polymorphism such as a AR gene polymorphism, a 5-alpha-reductase gene polymorphism, or an EDA2R gene polymorphism. Most preferably, the genotypes may be an AR CAG microsatellite, an AR GGN microsatellite, SNP rs6152, or SNP rs1385699.

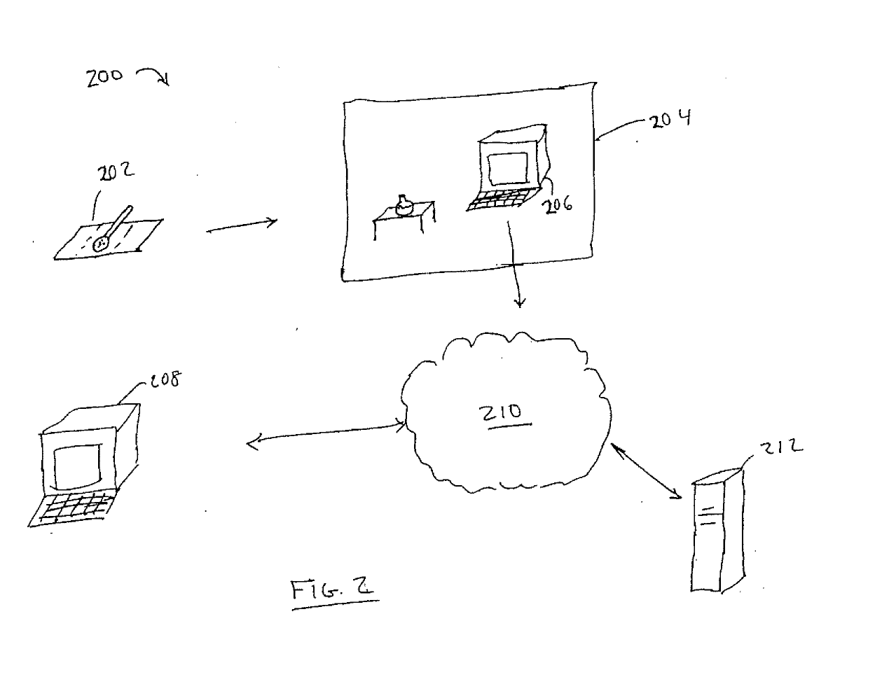

FIG. 2 schematically shows a system 200 for implementing an example embodiment of the present disclosure. A sample 202 of DNA from a subject may be sent to a lab 204. An analysis of the sample in accordance with one or more of the afore-mentioned procedures may then be conducted. Results of the analysis are for example compared with a database to generate an indication of the likelihood that the patient will be responsive to finasteride. The database may be dynamic in nature, continuously updated for statistical adaptation based on past finasteride treatment and response thereto, so that the database can adapt, or learn, from the patient pool and treatments over time, and in this manner become a better predictor of the responsiveness to the treatment. The database, or other entity or circuit or module capable of the adaptive scheme herein described, may reside in computer system 206 or separately therefrom. The outcome of the comparison and analysis can be forwarded to the subject's or caregiver's computer system 208, for example electronically by way of a network, such as the Internet, 210. Alternatively or in addition, the outcome of the comparison and analysis can be stored on a server 212 for accessing remotely by the subject or caregiver following proper authentication that may require reference to the unique identifier to preserve privacy.

It may also be possible to use a neural network to implement the testing system and method, to predict the likelihood that the patient will be responsive to of a drug such as finasteride based on the patient's genetic profile. In particular, the one or more media described in the embodiments above may comprise a neural network that has been trained with a plurality of reference profiles. One embodiment that uses a neural network according to the present disclosure might include: (a) constructing an N-layer neural network; (b) training the neural network with a set of reference profiles that may include, among other things, data set of patients' responsiveness to treatment with the drug for androgenetic alopecia, and genetic profiles of reference patients; (c) obtaining a genetic sample from the subject; (d) generating a genetic profile from the sample, the profile being a function of values associated with a prescribed set of human genes, or other information; (e) inputting the patient's profile into the neural network; and (f) generating a value or set of values from the neural network indicative of the patient's expected response to the drug treatment at a single or multiple dosages; and (g) providing the patient the drug treatment at the recommended dosage.

The above are exemplary modes of carrying out the invention and are not intended to be limiting. It will be apparent to those of ordinary skill in the art that modifications thereto can be made without departure from the spirit and scope of the invention as set forth in the following claims.

1. A method for prognosis of a patient's response to a drug for treating an androgenetic skin disorder, comprising:
obtaining a sample from the patient comprising one or more cells;
extracting genetic material from the sample;
generating a patient profile comprising a set of one or more patient score values, each patient score value being a function of one or more measurable characteristics selected from the group consisting of:
one or more genotypes within the genetic material, each genotype being an allele of a genetic polymorphism selected from the group consisting of (a) an androgen-related polymorphism, (b) an EDA2R gene polymorphism, and (c) a polymorphism having an allele in linkage disequilibrium with an allele of a genetic polymorphism selected from the group consisting of an androgen-related polymorphism and an EDA2R gene polymorphism;
a measured concentration of 5-alpha-reductase within the sample; and
a measurement of transcriptional activity of an 5-alpha-reductase gene within the sample; and
generating a prognosis as to whether or not the patient is likely to be responsive to the drug at a selected dosage, wherein the step comprises comparing one or more of the patient score values with reference information stored in one or more media.

2. The method of claim 1, wherein for each of the one or more genotypes, the genetic polymorphism is selected from the group consisting of an AR gene polymorphism, a 5-alpha-reductase gene polymorphism, and an EDA2R gene polymorphism.

3. The method of claim 2, wherein for each of the one or more genotypes, the genetic polymorphism is selected from the group consisting of an AR CAG microsatellite, an AR GGN microsatellite, an SNP rs6152, and an SNP rs1385699.

4. The method of claim 1, wherein the drug is an anti-androgen.

5. The method of claim 4, wherein the drug is a 5-alpha-reductase inhibitor.

6. The method of claim 5, wherein the drug is finasteride.

7. The method of claim 5, wherein the drug is selected from the group consisting of duasteride and FCE 28260.

8. The method of claim 4, wherein the drug is selected from the group consisting of spironolactone, flutamide, cyproterone acetate, nilutamide, and bicalutamide.

9. The method of claim 4, wherein the drug is a composition that inhibits the activity of the DHT or testosterone androgen pathway.

10. The method of claim 1, wherein the androgenetic skin disorder is androgenetic alopecia.

11. The method of claim 10,
wherein the androgenetic skin disorder is male androgenetic alopecia; and
wherein the drug is finasteride and the dosage is less than or equal to about 0.75 mg daily.

12. The method of claim 1, wherein the dosage is less than or equal to about 0.5 mg daily.

13. The method of claim 10,
wherein the androgenetic skin disorder is female androgenetic alopecia; and
wherein the drug is finasteride and the dosage is less than or equal to about 2.5 mg daily.

14. The method of claim 13, wherein the dosage is less than or equal to about 1.0 mg daily.

15. The method of claim 1, wherein the androgenetic skin disorder is selected from the group consisting of adult onset acne and hirsutism.

16. The method of claim 15, wherein the drug is selected from the group consisting of spironolactone, flutamide, cyproterone acetate, nilutamide, and bicalutamide.

17. The method of claim 1, wherein the sample from the patient comprises one or more cells selected from the group consisting of buccal cells, peripheral blood cells, or cells from the patient's integumentary system.

18. The method of claim 17, wherein the step of obtaining a sample from the patient comprises receiving through a mail or delivery service a sample collection kit comprising the sample.

19. The method of claim 17, wherein the sample from the patient comprises one or more scalp cells or hair stem cells, and wherein the step of obtaining a sample from the patient comprises receiving a biopsy from the patient comprising the one or more scalp cells or hair stem cells.

20. The method of claim 19, wherein the measurable characteristics are selected from the group consisting of the concentration of 5-alpha-reductase within the sample, and the transcriptional activity of the 5-alpha-reductase gene within the sample.

21. The method of claim 1, wherein the step of obtaining a genetic profile comprises hybridization to one or more allele-specific oligonucleotide probes.

22. The method of claim 21, wherein the one or more allele-specific oligonucleotide probes are anchored to a substrate selected from the group consisting of a nucleic acid microarray and beads.

23. The method of claim 1, wherein at least one of the patient score values is a function of two or more measurable characteristics.

24. The method of claim 23, wherein at least one of the patient score values is a function of three or more measurable characteristics.

25. The method of claim 1, wherein the step of generating a prognosis comprises:
generating a first comparison result or one or more other comparison results based on the step of comparing one or more of the patient score values with the reference information, the first and one or more other comparison results being mutually exclusive; and
determining that the patient is likely to be responsive to the drug if the first comparison result is generated.

26. The method of claim 25,
wherein the reference information comprises a numerical reference value corresponding to a first patient score value; and
wherein the step of comparing one or more of the patient score values with the reference information comprises comparing the first patient score value with the numerical reference value.

27. The method of claim 26,
wherein the first patient score value is selected from the group consisting of the length of an AR CAG microsatellite within the genetic material, and the length of an AR GGN microsatellite within the genetic material;
wherein the numerical reference value is 20; and
wherein the first comparison result is generated when the first patient score value is less than the numerical reference value.

28. The method of claim 26,
wherein the first patient score value is the sum of the length of an AR CAG microsatellite within the genetic material plus the length of an AR GGN microsatellite within the genetic material;
wherein the numerical reference value is 40; and
wherein the first comparison result is generated when the first patient score value is less than the numerical reference value.

29. The method of claim 28, wherein the AR CAG microsatellite and the AR GGN microsatellite are located on the active X chromosome.

30. The method of claim 26,
wherein the first patient score value is the longer of (a) the length of an AR CAG microsatellite within the genetic material or (b) the length of an AR GGN microsatellite within the genetic material;
wherein the numerical reference value is 20; and
wherein the first comparison result is generated when the first patient score value is less than the numerical reference value.

31. The method of claim 26,
wherein a first chromosome score is a function of one or more quantities selected from the group consisting of: the length of an AR CAC microsatellite within a first X chromosome within the genetic material, the length of an AR GGN microsatellite within the first X chromosome within the genetic material, and the length of a TA dinucleotide microsatellite within a 5-alpha-reductase gene within the first X chromosome within the sample;
wherein a second chromosome score is a function of one or more quantities selected from the group consisting of: the length of an AR CAG microsatellite within a second X chromosome within the genetic material, the length of an AR GGN microsatellite within the second X chromosome within the genetic material, and the length of a TA dinucleotide microsatellite within a 5-alpha-reductase gene within the second X chromosome within the sample;
wherein the second X chromosome is homologous to the first X chromosome; and
wherein the first patient score value is selected from the group consisting of (a) the greater of the first chromosome score and the second chromosome score, (b) the lesser of the first chromosome score and the second chromosome score, (c) the sum of the first chromosome score and the second chromosome score, and (d) the average of the first chromosome score and the second chromosome score.

32. The method of claim 25,
wherein the reference information comprises aggregate data from a plurality of reference profiles, each reference profile corresponding to a reference patient who has been administered the drug, and from whom a reference sample has been collected,
wherein each reference profile comprises:
an indication of the corresponding reference patient's responsiveness or non-responsiveness to the drug; and
a set of one or more reference values, each reference value being a function of one or more measurable reference characteristics selected from the group consisting of:
one or more reference genotypes within genetic material obtained extracted from the reference sample, each reference genotype being an allele of a genetic polymorphism selected from the group consisting of (a) an androgen-related polymorphism, (b) an EDA2R gene polymorphism, and (c) a polymorphism having an allele in linkage disequilibrium with an allele of a genetic polymorphism selected from the group consisting of an androgen-related polymorphism and an EDA2R gene polymorphism;
a measured concentration of 5-alpha-reductase within the a reference sample; and
a measurement of transcriptional activity of an 5-alpha-reductase gene within the reference sample; and
wherein the plurality of reference profiles comprise:
a first reference profile corresponding to a first reference patient whose reference profile is indicative of responsiveness to the drug; and
a second reference profile corresponding to a second reference patient whose reference profile is indicative of non-responsiveness to the drug.

33. The method of claim 32, wherein the one or more media comprise an electronic database into which the plurality of reference profiles have been stored.

34. The method of claim 32,
wherein the one or more media comprise a neural network that has been trained with the plurality of reference profiles;
wherein the step of generating a prognosis comprises:
inputting the patient profile into the neural network; and
generating a value or set of values from the neural network indicative of the patient's expected response to the drug treatment at a single or multiple dosages.

35. The method of claim 32,
wherein the aggregate data comprises a set of statistical values describing a statistical distribution based on the reference profiles; and
wherein the first comparison result is generated when one or more of the patient score values meets one or more statistical criteria based on the statistical distribution.

36. The method of claim 32,
wherein the androgenetic skin disorder is androgenetic alopecia; and
wherein for each reference profile, the indication of the corresponding reference patient's responsiveness or non-responsiveness to the drug comprises an assessment selected from the group consisting of.
a physician's assessment of the responsiveness or non-responsiveness of the drug using a standardized scale; and
an assessment of one or more criteria selected from the group consisting of hair growth measurement, hair thickness measurement, selected hair diameter measurement, average hair diameter measurement, and average hair length measurement.

37. The method of claim 1, further comprising the step of writing a prescription or administering the drug to the patient.

38. The method of claim 1, further comprising the step of recording the prognosis on a medium.

39. The method of claim 38, further comprising the step of delivering or transmitting the prognosis indication to the patient.

40. A system for generating a prognosis of a patient's response to a drug for treating an androgenetic skin disorder, comprising:
means for storing a patient profile comprising a set of one or more patient score values, each patient score value being a function of one or more measurable characteristics selected from the group consisting of:
one or more genotypes within the genetic material, each genotype being an allele of a genetic polymorphism selected from the group consisting of (a) an androgen-related polymorphism, (b) an EDA2R gene polymorphism, and (c) a polymorphism having an allele in linkage disequilibrium with an allele of a genetic polymorphism selected from the group consisting of an androgen-related polymorphism and an EDA2R gene polymorphism;
a measured concentration of 5-alpha-reductase within the sample; and
a measurement of transcriptional activity of an 5-alpha-reductase gene within the sample;
means for storing reference information comprising aggregate data from a plurality of reference profiles, each reference profile corresponding to a reference patient who has been administered the drug, and from whom a reference sample has been collected,
wherein each reference profile comprises:
an indication of the corresponding reference patient's responsiveness or non-responsiveness to the drug; and
a set of one or more reference values, each reference value being a function of one or more measurable reference characteristics selected from the group consisting of:
one or more reference genotypes within genetic material obtained from the reference sample, each reference genotype being an allele of a genetic polymorphism selected from the group consisting of (a) an androgen-related polymorphism, (b) an EDA2R gene polymorphism, and (c) a polymorphism having an allele in linkage disequilibrium with an allele of a genetic polymorphism selected from the group consisting of an androgen-related polymorphism and an EDA2R gene polymorphism;
a measured concentration of 5-alpha-reductase within the reference sample; and
a measurement of transcriptional activity of an 5-alpha-reductase gene within the reference sample; and
wherein the plurality of reference profiles comprise:
a first reference profile corresponding to a first reference patient whose reference profile is indicative of responsiveness to the drug; and
a second reference profile corresponding to a second reference patient whose reference profile is indicative of non-responsiveness to the drug;
means for comparing one or more of the patient score values with the reference information; and
means for generating a prognosis as to whether or not the patient is likely to be responsive to the drug at a selected dosage.

41. A composition of matter for treatment of an androgenetic skin disorder, comprising:
a drug in a form suitable for administration to a human; and
a medium packaged with the drug comprising:
an indication that the drug is for treatment of the androgenetic skin disorder;
an indication that the drug is for use by a person who has one or more patient characteristics, wherein each of the one or more patient characteristics is selected from the group consisting of:
one or more genotypes within the person's genetic material, each genotype being an allele of a genetic polymorphism selected from the group consisting of (a) an androgen-related polymorphism, (b) an EDA2R gene polymorphism, and (c) a polymorphism having an allele in linkage disequilibrium with an allele of a genetic polymorphism selected from the group consisting of an androgen-related polymorphism and an EDA2R gene polymorphism;
a measured concentration of 5-alpha-reductase within a sample taken from the person; and
a measured transcriptional activity of an 5-alpha-reductase gene within a sample taken from the person.

42. The method of claim 41, wherein the drug is finasteride, and wherein the medium farther comprises an indication that the person is female.

43. The method of claim 42, wherein the androgenetic skin disorder is adult onset acne.

44. The method of claim 41, wherein the drug is finasteride, and wherein the medium further comprises:
an indication that the person is male; and
an indication of recommended dosage, wherein the recommended dosage is an amount less than or equal to about 0.75 milligrams daily.


SEQUENCE LISTING (4 sequences)

SEQ ID NO: 1 - DNA, 19 bp, Homo sapiens
gtgcgcgaagtgatccaga

SEQ ID NO: 2 - DNA, 23 bp, Homo sapiens
gtttcctcatccaggaccaggta

SEQ ID NO: 3 - DNA, 22 bp, Homo sapiens
ccgcttcctcatcctggcacac

SEQ ID NO: 4 - DNA, 21 bp, Homo sapiens
gccgccagggtaccacacatc